(19) United States
(12) Patent Application Publication
(10) Pub. No.: US 2021/0169363 A1
(43) Pub. Date: Jun. 10, 2021

(54) DEVICE AND METHOD FOR DETERMINING THE IMPEDANCE ON A TOOTH

(71) Applicant: Philipps-Universitat Marburg, Marburg (DE)

(72) Inventors: Michael Gente, Cölbe (DE); Matthias Willamowski, Mertloch (DE)

(21) Appl. No.: 16/762,753

(22) PCT Filed: Nov. 9, 2018
(86) PCT No.: PCT/EP2018/080717

§ 371 Date: May 8, 2020

(30) Foreign Application Priority Data
Nov. 10, 2017 (DE) 10 2017 126 496.5

(51) Int. Cl. A61B 5/0534; A61B 5/00; A61C 17/06

(52) U.S. Cl. CPC A61B 5/0534; A61B 5/7405; A61B 2562/0209; A61B 5/7455; A61C 17/06; A61B 5/742

(57) ABSTRACT

The present invention relates to an apparatus and a method for determining impedance (AC resistance) on a tooth. The impedance is determined by measuring the voltage at a given current intensity or by measuring the current intensity at a given voltage. Determining the impedance on a tooth is used for diagnosing tooth decay.

The present invention relates to an apparatus and a method for determining impedance on a tooth. The impedance on a tooth can be used to diagnose tooth decay (caries, carious lesion, carious change; plural: caries).

Tooth decay is a disease of the tooth, in which the dental hard tissue, i.e., the enamel and/or the dentin, is damaged. The dental hard tissue is also referred to as hard substance or dental hard substance. Decalcification, discoloring, cavitations (collapse of the dental hard tissue) and lesions into the pulpa are symptoms of caries.

If the caries only affects the enamel it is referred to as enamel caries while if the caries also affects the dentin it is referred to as dentin caries. If the caries affects the chewing surface of a tooth it is referred to as occlusal caries and if the caries affects the surfaces of a tooth adjoining the neighboring teeth it is referred to as approximal caries.

Since untreated caries leads to the loss of the affected tooth it is essential to already diagnose caries at the earliest possible stage. At the same time, false positive results within the scope of caries diagnostics should be avoided where possible in order to prevent an over treatment. Thus a goal of caries diagnostics lies in specificity and sensitivity that are as high as possible in order to be able to treat the patient appropriately.

Caries diagnostics on the tooth can be difficult since small carious changes cannot be distinguished visually from discolorations of the enamel during the clinical examination. Therefore, there are the following two risks: Firstly, there is the risk of over treatment if a point that is only discolored is falsely diagnosed as caries and therefore drilled open and filled; secondly, there is the risk of under treatment if caries is not identified and therefore remains untreated.

Reliable diagnosis or reliable exclusion of caries is particularly difficult at the approximal surfaces of the teeth, i.e., at the teeth surfaces adjoining the neighboring teeth. The interdental space which, with individual differences, can be very narrow is situated between the approximal surfaces of two adjacent teeth. As the interdental space becomes smaller, the relevant approximal surfaces become more difficult to access for a visual examination. Approximal caries is very easily overlooked, especially in the case of very narrow interdental spaces.

### STATE OF THE ART

The following apparatuses or methods are used for diagnosing caries:

Visual diagnosis: The cleaned and dried tooth is examined by observation and under good illumination for discoloring and cavitation (collapse of the dental hard tissue) using a dental mirror.

Tactile probing: The cleaned and dried tooth is sensed by a dental probe.

Fiber-optic transillumination (FOTI, diaphanoscopy): The dental hard tissue is transilluminated using a cold light probe. Here, different behavior of healthy and carious dental hard tissue when diffracting light is used. Carious substance becomes identifiable as a dark shadow on account of the light intensity loss. This allows good diagnosis of dentin caries in particular.

X-ray examination: X-ray examinations using conventional or digital technology are performed by means of bite wings. This allows a good diagnosis of enamel caries on the approximal surfaces, i.e., the tooth surfaces at which the teeth of a row of teeth are in contact, in particular.

Laser-assisted caries diagnostics (laser fluorescence measurement): The light of a laser fluorescence device at a wavelength of 650 nm is resorbed by both organic and inorganic substances. Laser fluorescence devices consist of at least one light source and an optical unit, which have a size typical for the design. Since a carious lesion in the dental hard substance is excited to fluoresce by the employed laser, caries can be deduced if fluorescence is present. This method is particularly well-suited for diagnosing occlusal caries.

Determining the AC resistance on the tooth (impedance measurement): The impedance of the dental hard substance is determined in this method using a suitable measuring device. In the case of caries, the electrical conductivity is significantly increased in comparison with healthy dental hard substance and hence the impedance is significantly reduced. Since enamel has a substantially higher impedance than dentin, this method can be used, in particular, to diagnose carious lesions of the enamel. Should caries not have led to cavitation yet and therefore be identifiable neither visually nor by means of a tactile probe, then the impedance measurement is a very well suited method.

Devices operating with alternating currents are used for determining the impedance. These measuring devices comprise at least a reference electrode, a measuring electrode and a measuring unit. The reference electrode is placed at any position in the oral cavity. The measuring electrode is used to sense the tooth to be examined. The impedance drops as soon as a carious change is contacted by the measuring electrode. This change in impedance is registered by the measuring unit and indicated to the examiner, for example by way of an acoustic warning signal or an optical display. The pointer of an electrical AC resistance measuring device can be used as an optical display, or else a luminous display which changes color, for example from green to red, when the electrical resistance changes. By way of example, the luminous display can be an LED scale with diodes of different colors.

Predominantly the occlusal surfaces with their fissures and small pits are examined when sensing the surface of the tooth to be examined using the measuring electrode. If the measuring electrode contacts a carious point then the impedance is lowered because the enamel of the tooth is porous at carious points.

The disadvantages of the prior art are the following:

Visual diagnosis: The interdental spaces and hence the approximal surfaces of a tooth are not accessible or only accessible very poorly for visual diagnosis. Thus, approximal caries is often overlooked within the scope of the visual diagnosis. The meaningfulness of this clinical examination process depends very strongly on the experience and the color perception of the dentist. Physiological discolorings of the tooth can be misdiagnosed as caries.

Tactile probing: Unskilled action within the scope of tactile probing can lead to the collapse of the poorly mineralized enamel or to other damage on the tooth. Moreover, in comparison with the visual diagnosis, it only provides insubstantially more insight. For these reasons, tactile probing is now considered obsolete.

Fiber-optic transillumination: Physiological tooth discolorations which may occur independently of caries falsify the results of the fiber-optic transillumination such that false positive diagnoses are made.

X-ray examination: Each examination represents radiation exposure, which should be kept as low as possible as a matter of principle. Therefore, there must be indication justifying an x-ray examination, particularly in the case of children and pregnant patients. Since the patient must not move in the slightest while an x-ray recording is taken, x-ray recordings are often not performable in the case of relatively small children or persons with restricted cognitive functions. Dentin caries can easily be overlooked during the x-ray examination since changes can only be diagnosed as carious once demineralization has significantly advanced into the dentin. Occlusal caries is only identifiable in an x-ray image once the carious lesions are already so deep that they reach as far as the dentin. Since x-rays are absorbed and scattered by fillings already present, caries directly below or next to an already present filling cannot always be identified within the scope of an x-ray examination.

Laser-assisted caries diagnostics: This method can hardly be used to diagnose approximal caries since the employed laser fluorescence devices cannot be placed between two teeth (i.e., toward the approximal surfaces) on account of their size that is typical for the design. Physiological tooth discolorations which may occur independently of caries falsify the results of the laser-assisted caries diagnostics. Laser fluorescence devices are expensive both in terms of procurement and operation.

Determining the AC resistance on the tooth (impedance measurement): The composition and the amount of saliva on the tooth to be examined and in the oral cavity influence the electrical conductivity and hence the impedance. The impedance drops if a lot of saliva is situated on the tooth to be examined, even if no caries is present. Thus, false positive findings are made since the electrical current in this case flows to the oral cavity not only through the tooth but also through the saliva film on the tooth surface; thus, there often is an unidentified electrical shunt. Likewise, the electrolyte content of the individual saliva, which differs from patient to patient, may influence impedance.

This disadvantage exists, in particular, in the measuring devices currently used for measuring impedance. The measuring devices currently used for impedance measurements are lacking in terms of reproducibility of the measurement results, which may confuse the dentist. The problem exists, in particular, in the case of repeated measurements for monitoring progress.

Since caries preferably arises in fissures and small pits on the tooth surface, measuring electrodes with dimensions that are too large cannot reliably detect initial carious changes.

The measuring devices currently used for measuring impedance have rod-shaped, cylindrical or wire-shaped measuring electrodes. On account of this electrode form, the measuring electrodes from the prior art are not suitable for sensing the approximal surfaces of the teeth. Consequently, approximal caries cannot be diagnosed using the currently known measuring devices.

### PROBLEM

The present invention should provide an apparatus and a method that improve the determination of the impedance on a tooth for diagnosing caries. In particular, the influence of the saliva present in the oral cavity should be reduced. Moreover, the reproducibility of the measurements when monitoring progress should be improved in order to reduce dentist confusion and consequently reduce incorrect diagnoses. Moreover, diagnosing caries in fissures and small pits on the tooth surface should be improved in order also to reliably identify early stages of caries. Additionally, diagnosing approximal caries using suitable measuring electrodes should be facilitated.

### SOLUTION TO THE PROBLEM

The problem according to the invention is solved by claim 1 (apparatus) and claim 14 (method). In particular, the problem according to the invention is solved by the following developments over the prior art:
Using an insulating gel 9 with an electrical resistivity of ρ>500 Ω·m for avoiding the bothersome influence of the saliva.
Improving the measuring electrode 5 by coupling the latter to a compensating electrode 6: As a result, the current that would have been discharged laterally from the measuring electrode 5 via the tooth surface is guided via the compensating electrode 6. The compensating electrode 6 acts as a shield for the measuring electrode 5.
Adapting the measuring electronics in the measuring unit 2: Stable measured values are obtained, even in the case of very small currents, as a result of carrying out the alternating current measurement using the lock-in technique. This allows stable measured values to be obtained in the case of currents with a magnitude of between 10 nA and 2 μA.
Performing the measurement according to the carrier frequency measuring technique principle.
The measuring unit 2 provides a defined sinusoidal voltage by means of a function generator. Preferably, this sinusoidal voltage has a frequency of 600 Hz and a mean voltage of approximately 70 mV Uᵣₘₛ. By way of an isolation amplifier in each case, this voltage is applied both to the measuring electrode 5 and to the compensating electrode 6.

Thus, the apparatus 1 according to the invention and the method according to the invention determine, in an improved fashion, the impedance of the dental hard substance to be examined. In the case of caries, the impedance is reduced at the affected point of the dental hard substance. Ohm's law is applied for determining the impedance. The impedance is determined by way of a calculation from the measured values of the current flow between the measuring electrode 5 and the reference electrode 4 at a given voltage. Alternatively, the impedance can also be determined by way of a calculation from the measured values of the voltage at a given current flow between the measuring electrode 5 and the reference electrode 4.

In order to reduce the influence of the saliva present in the oral cavity, the saliva present at the tooth to be examined is initially removed and an insulating gel 9 is subsequently applied to said tooth when determining the impedance, according to the invention. This insulating gel 9 can only have a low electrical conductivity and must be non-toxic and cheap to produce. Preferably, the insulating gel 9 has an electrical resistivity of ρ>500 Ω·m. The viscosity of the insulating gel 9 should be so high that it adheres to the tooth to be examined. Advantageously, it has a coloring that differs from all other structures in the oral cavity so that it is more easily identifiable. Whether the insulating gel 9 was applied in sufficient quantities and at the envisaged points can therefore easily be determined by way of a visual control. A gel-type preparation of 0.9 g galactose polymer agar (or agar-agar) per 100 ml distilled water+1 ml dye was found to be particularly advantageous for the insulating gel 9. Advantageously, a blue food colorant such as, e.g., anthocyanin (E163), brilliant blue FCF (E133), indigo carmine (E132) or patent blue V (E131) is used as a dye.

Alternatively, the insulating gel 9 can also be prepared from gelatin or starch.

In one embodiment, the insulating gel 9 is embodied in the form of flexible bags or pads, which can be pressed onto the tooth to be examined and which match the tooth surface. These bags or pads are advantageous in that they can be stored more easily and can be taken from individual packaging when necessary.

The apparatus 1 according to the invention comprises at least the following further components:

A measuring unit 2: The measuring unit 2 comprises at least one functional generator, an isolation amplifier, a lock-in amplifier, a voltage indicator, an evaluation unit and an output means for an acoustic, optical and/or haptic signal. The measuring unit 2 is connectable to the reference electrode 4, the measuring electrode 5 and the compensating electrode 6. Advantageously, this connection is a plug-in connection so that the electrodes 4, 5 and 6 can be cleaned after use in a patient and can be reused.

By way the functional generator, the measuring unit 2 provides the AC voltages that are applied to the electrodes 4, 5 and 6 connected to the measuring unit. The measuring unit 2 measures the current intensity that flows from the measuring electrode 5 to the reference electrode 4 through the tooth. From the resulting current intensity, the evaluation unit of the measuring unit 2 determines the level of impedance at a given voltage. In an alternative embodiment, the measuring unit 2 measures the voltage at a given current intensity. In any case, the evaluation unit of the measuring unit 2 captures the level and the change in impedance at at least two different positions on the tooth to be examined.

Thus, the evaluation unit of the measuring unit 2 registers a change in the impedance, processes and evaluates the measurement results and provides the examiner with suitable feedback via the output means, for example an acoustic warning signal and/or an optical indication and/or a haptic indication. The output means of the measuring unit 2 is connectable to the measuring unit 2.

The currents measured for determining the impedance of a tooth are very small. A lock-in amplifier is used for better evaluation thereof. This avoids faults in the case of small measurement currents. As a result of the lock-in amplifier and the lock-in technique possible therewith, stable measured values are obtained even in the case of very small currents ranging from 10 nA to 2 μA.

A handle 3: The handle 3 is embodied in such a way that the apparatus 1 with the measuring unit 2 can be held during the examination and the electrodes 4, 5 and 6 of the apparatus 1 can be placed in the oral cavity or on the tooth to be examined. The handle 3 has a surface made of an electrically insulating material, for example a plastic such as polycarbonate or polyethylene.

The measuring unit 2 can be integrated into the handle 3 such that the handle 3 and the measuring unit 2 only require a common housing. In this advantageous embodiment, the handle 3 is designed in such a way that the advantageous plug-in connections of the electrodes 4, 5 and 6 in the measuring unit 2 are facilitated.

A reference electrode 4: It consists of an electrically conductive material and is placed at any point in the oral cavity. It is connected to the measuring unit 2 by way of an electrical conductor. In a preferred embodiment, the reference electrode 4 is embodied to be connectable to the measuring unit 2 by way of a plug. As a result, the reference electrode 4 can easily be changed after the use in a patient. The reference electrode 4 is embodied in such a way that it can easily be cleaned and sterilized.

A measuring electrode 5: It consists of an electrically conductive material. It is connected to the measuring unit 2 by way of an electrical conductor. In a preferred embodiment, the measuring electrode 5 is embodied to be connectable to the measuring unit 2 by way of a plug. As a result, the measuring electrode 5 can easily be changed after the use in a patient. The measuring electrode 5 is embodied in such a way that it can easily be cleaned and sterilized.

The measuring electrode 5 is disposed relative to the reference electrode 4 in such a way that an alternating current can flow between the measuring electrode 5 at a first position on a tooth to be examined and the reference electrode 4.

The surface of the tooth to be examined is sensed using the measuring electrode 5. Consequently, the measuring electrode 5 is moved to at least one further position on a tooth to be examined. At the point where the measuring electrode 5 contacts the tooth it displaces the insulating gel 9 from the tooth surface together with the compensating electrode 6 and the circuit is closed by way of the reference electrode 4. If this point on the tooth is not changed by caries, then impedance is very high since intact enamel and intact dentin have a high electrical resistance of more than 600 kΩ. As soon as the measuring electrode 5 touches the region of carious change, the impedance drops to below 480 kΩ. This change in the impedance is determined, processed and evaluated by the measuring unit 2 by way of the measurement of the current flow and output to the examiner.

A compensating electrode 6: It consists of an electrically conductive material. It is connected to the measuring unit 2 by way of an electrical conductor. In a preferred embodiment, the compensating electrode 6 is embodied to be connectable to the measuring unit 2 by way of a plug. As a result, the compensating electrode 6 can easily be changed after the use in a patient. The compensating electrode 6 is embodied in such a way that it can easily be cleaned and sterilized.

The compensating electrode 6 is at the same electrical potential as the measuring electrode 5. Consequently, it has the same electrical potential as the measuring electrode 5. The compensating electrode 6 is structurally coupled to the measuring electrode 5. It serves to shield the measuring electrode 5.

The measuring electrode 5 and the compensating electrode 6 are not electrically interconnected but separated from one another by an insulating layer 7 (insulating layer, insulator). Thus, an insulating layer 7 is applied between the measuring electrode 5 and the compensating electrode 6.

This structural arrangement of mechanical coupling with simultaneous electrical insulation ensures that the same electrical potential as at the measuring electrode 5 is present in the insulating gel 9, which surrounds the measuring electrode 5 and the compensating electrode 6. This ensures that currents flowing over the tooth surface only originate from the compensating electrode 6 and not from the measuring electrode 5. The compensating electrode 6 therefore causes the enamel at the point on the tooth to be examined and the applied insulating gel 9 to have the same electrical potential as the measuring electrode 5. This prevents the creation of an electric field which generates a current flow from the measuring electrode 5 to the oral cavity and hence to the reference electrode 4 via the tooth surface. Since this unwanted current flow reduces the reproducibility and the accuracy of the impedance determinations it is essential that this be suppressed.

An insulating layer 7: The insulating layer 7 is applied between the measuring electrode 5 and the compensating electrode 6 in such a form that these two electrodes are electrically insulated from one another and no current can flow between said two electrodes. The insulating layer 7 consists of a suitable insulating material such as, e.g., a plastic, for example polycarbonate or polyethylene. Together with the insulating gel 9, the insulating layer 7 prevents currents flowing over the tooth surface from originating from the measuring electrode 5. This significantly improves the reproducibility and accuracy of the measurements.

A schematic illustration of the apparatus according to the invention is shown in FIGS. 1 and 2.

The method according to the invention comprises the following steps:
Using processes known from the prior art, the tooth to be examined is made as dry as possible in order to prevent the individually different influences of the saliva. By way of example, an air blower can blow on the tooth for the purposes of drying the saliva film. After drying, e.g., cotton rolls or a dental dam keep new saliva away.
The tooth to be examined is wetted by the insulating gel 9 according to the invention. Thus, for example, this tooth is wetted using a gel-like preparation made of 0.9 g agar-agar per 100 ml distilled water+1 ml dye. Wetting is implemented over the entire visible surface of the tooth or else only at the points to be examined.
This creates standardized and comparable measurement conditions. In particular, the admission of natural, electrically well conducting saliva is prevented, said saliva potentially disturbing the measurement of the current flow and hence the determination of the impedance as a shunt to the oral cavity and hence to the reference electrode 4.
The reference electrode 4 is placed at any point in the oral cavity without coming into direct contact with the measuring electrode 5 and the compensating electrode 6. This point must not have any pathological changes (e.g., inflammations, ulcers or tumorous changes). This point is not treated prior to the application of the reference electrode 4 and consequently wetted by saliva. Since the electrical resistance of the tissue between the tooth to be examined and the reference electrode 4 is negligibly small in comparison with the electrical resistance of the dental hard substance, the spatial distance between the reference electrode 4 and the examined tooth is negligible. Therefore, the position in the oral cavity at which the reference electrode 4 is placed is immaterial.
The measuring electrode 5 is applied to the point of the tooth to be examined and brought into contact with the latter. Thus, the surface of the tooth to be examined is sensed using the measuring electrode 5. In the process, the measuring electrode 5 displaces the insulating gel 9 at the contact point to the tooth; said insulating gel consequently forms a wall-like, insulating layer around the measuring electrode 5.
As a result of the mechanical coupling of the compensating electrode 6 to the measuring electrode 5, the compensating electrode 6 is also simultaneously applied to the point of the tooth to be examined and the insulating gel 9 forms a wall-like, insulating layer around the compensating electrode 6. As it were, the measuring electrode 5 and the compensating electrode 6 are immersed in the insulating gel 9 and consequently come into contact with the tooth surface. This closes the circuit between the measuring electrode 5 and the reference electrode 4.
An electrical AC voltage U is applied between the measuring electrode 5 and the reference electrode 4. By way of example, this AC voltage U has a frequency between 600 and 2000 Hz. Preferably, its maximum amplitude is approximately 200 mV. Preferably, its mean amplitude Uᵣₘₛ is approximately 70 mV.
An AC voltage is likewise applied to the compensating electrode 6, with the amplitude, frequency and phase angle thereof corresponding to the voltage at the measuring electrode 5.
The measuring unit 2 now measures the current which flows through the measuring electrode 5 at a constant voltage and flows through the tooth to the reference electrode 4. This current flow is used as a measured value, from which the measuring unit 2 determines impedance. Small currents correspond to a large impedance; large currents correspond to a small impedance.
Alternatively, the measuring unit 2 measures the voltage applied to the measuring electrode 5 at a constant current intensity. This voltage is used as a measured value, from which the measuring unit 2 determines impedance.
The impedance Z is determined on the basis of Ohm's law Z=U/I.

As a result of the arrangement of the compensating electrode 6 according to the invention and the use of the insulating gel 9 according to the invention, the current flow from the measuring electrode 5 can only lead into the tooth to be examined via the tip of the measuring electrode 5 and cannot lead laterally via the tooth surface to the gingiva and hence into the oral cavity to the reference electrode 4. Consequently, disturbing electric fields and unwanted current flows are prevented. This achieves a substantially better reproducibility and accuracy, as a result of which more precise progress examinations are facilitated. Likewise, substantially improved specificity and sensitivity are achieved in comparison with the determination of the impedance from the prior art.

### FURTHER EMBODIMENTS

In one embodiment, the reference electrode 4 is embodied as a bent, stainless wire.

In one embodiment, the reference electrode 4 consists of a metal, such as titanium, silver or iron, or of a metal alloy or, preferably, of stainless steel; in another embodiment, the reference electrode 4 consists of carbon.

In one embodiment, the measuring electrode 5 consists of a metal, such as titanium, silver or iron, or of a metal alloy or, preferably, of stainless steel; in another embodiment, the measuring electrode 5 consists of carbon.

Advantageously, an AC voltage at a frequency of approximately 600 Hz and a maximum amplitude of approximately 200 mV is applied to the measuring electrode 5. Hence, the mean amplitude Uᵣₘₛ is approximately 70 mV.

In one embodiment, the compensating electrode 6 consists of a metal, such as titanium, silver or iron, or of a metal alloy or, preferably, of stainless steel; in another embodiment, the compensating electrode 6 consists of carbon.

In one embodiment, the measuring electrode 5 is mounted resiliently in order to compensate the irregularities of the tooth surface (fissures, small pits).

In a preferred embodiment, the measuring electrode 5 is embodied as an elongate electrode with a tapering end running towards a tip. This even allows measurements to be performed in the smallest depressions in the tooth surface. Likewise, this allows measurements to be carried out at very small changes, suspected to be carious, in fissures or small pits in the tooth surface. Measuring electrodes 5 with diameters of less than 1.5 mm were found to be particularly advantageous to this end.

In one embodiment, the measuring electrode 5 and the compensating electrode 6 mechanically coupled thereto are embodied as a planar film. This also allows measurements to be performed in the interdental spaces at the approximal surfaces of the tooth to be examined. For the carrier material of the film, use can be made of plastic or any other substance that has a very low electrical conductivity, for example polycarbonate or polyethylene. Thus, this carrier material acts as an insulating layer 7 in this embodiment. The carrier material for the film is preferably transparent so that the dentistry staff can identify the tooth surface through the film. The measuring electrode 5 and the compensating electrode 6 are worked into this carrier material (i.e., the insulating layer 7). In this case, the measuring electrode 5 and the compensating electrode 6 consist of an abrasion-stable material, for example a metal such as titanium, silver or iron, or of a metal alloy or, preferably, of stainless steel, or of carbon.

In one embodiment, the measuring electrode 5 embodied as a planar film and the compensating electrode 6 mechanically coupled thereto have a reinforced edge. This simplifies the placement of the measuring electrode 5 and compensating electrode 6, embodied as planar film, between two teeth (i.e., in the interdental region). Likewise, this prevents crumpling of the measuring electrode 5 and compensating electrode 6 embodied as planar film.

In one embodiment, the reinforced edge of the planar film is embodied as a compensating electrode 6. In another embodiment, the reinforced edge of the planar film is embodied as a measuring electrode 5.

In one embodiment, the planar film has two measuring electrodes 5 and two compensating electrodes 6, which are controllable separately from one another by the measuring unit 2. As a result, following the placement of the planar film in the interdental space between two adjacent teeth, the one tooth can be examined first using the first measuring electrode 5 and the first compensating electrode 6, followed by the other tooth using the second measuring electrode 5 and the second compensating electrode 6. This is advantageous in that the film only has to be placed into the interdental region once when examining the approximal surfaces of two adjacent teeth. This is advantageous for the patient and the dentist, especially in the case of narrow interdental regions.

In one embodiment, the compensating electrode 6 is embodied as a cylindrical tube; in another embodiment, it is embodied as a cylindrically wound wire. In both embodiments, the compensating electrode 6 surrounds the centrally disposed measuring electrode 5 and both are electrically insulated from one another by way of an insulating layer 7.

In a further embodiment, the compensating electrode 6 is disposed relative to the measuring electrode 5 in such a way that they are axially displaceable with respect to one another. By way of example, this is achieved by the installation of a resilient element 8 in the measuring electrode 5 or in the compensating electrode 6. Advantageously, the externally disposed compensating electrode 6 is coupled via a spring 8 to the centrally disposed measuring electrode 5. Here, it is essential for these two electrodes to be electrically insulated from one another by an insulating layer 7. The following advantage is achieved by this arrangement: If the measuring electrode 5 is pressed into a fissure or a small pit in the tooth to be examined (i.e., where caries preferably arises), then the tip of the compensating electrode 6 follows the tip of the measuring electrode 5 and both electrodes are applied close to the tooth surface. As a result, incorrect measurements and measurement variations are reduced.

In one embodiment, the electrodes 4, 5 and 6 are embodied as single use electrodes. In this case, the electrodes 4, 5 and 6 are advantageously connected to the measuring unit 2 by way of a plug-in connection.

In a preferred embodiment, the measuring unit 2 is integrated into the handle 3 such that the handle 3 and the measuring unit 2 only require a common housing.

### KEY TO THE FIGURES

FIG. 1 shows a schematic illustration of the apparatus 1 according to the invention in a longitudinal section: The measuring unit 2 and the handle 3 are disposed independently of one another. The measuring electrode 5 and the compensating electrode 6 are coupled to one another and placed on the tooth to be examined (illustrated schematically). The compensating electrode 6 has a tube-shaped (cylindrical) embodiment at the contact point to the tooth and consequently shields the measuring electrode 5. The reference electrode 4 is placed at any position in the oral cavity. The insulating gel 9 has not yet been applied to the surface of the tooth.

FIG. 2 shows a schematic illustration of the apparatus 1 according to the invention in a longitudinal section: The measuring unit 2 is integrated into the handle 3, and so only one common housing is required. The measuring electrode 5 and the compensating electrode 6 are coupled to one another and placed on the tooth to be examined (illustrated schematically). The compensating electrode 6 has a tube-shaped (cylindrical) embodiment at the contact point to the tooth and consequently shields the measuring electrode 5. The reference electrode 4 is placed at any position in the oral cavity. The insulating gel 9 has been applied to the entire surface of the tooth to be examined. At the point of contact of the measuring electrode and compensating electrode 5 and 6 with the tooth, these displace the insulating gel 9. As a result, the insulating gel 9 forms a wall-like edge around the measuring electrode 5 and around the compensating electrode 6.

FIG. 3 shows a schematic illustration of the apparatus 1 according to the invention in a longitudinal section: The measuring unit 2 is integrated into the handle 3, and so only one common housing is required. The measuring electrode 5 and the compensating electrode 6 are coupled to one another and placed on the tooth to be examined (illustrated schematically). The compensating electrode 6 is embodied as a cylindrically wound wire at the contact point to the tooth and consequently shields the measuring electrode 5. The reference electrode 4 is placed at any position in the oral cavity. The insulating gel 9 has not yet been applied to the surface of the tooth.

FIG. 4 shows a schematic illustration of the apparatus 1 according to the invention in a longitudinal section: The measuring unit 2 is integrated into the handle 3, and so only one common housing is required. The measuring electrode 5 and the compensating electrode 6 are coupled to one another and placed on the tooth to be examined (illustrated schematically). The measuring electrode 5 is resiliently mounted using a resilient element 8. As a result, the measuring electrode 5 has an axially displaceable arrangement in relation to the compensating electrode 6. A spring is provided as a resilient element 8. If the measuring electrode 5 is pressed into a fissure or a small pit in the tooth to be examined (i.e., where caries preferably arises), then the tip of the compensating electrode 6 follows the tip of the measuring electrode 5 and both electrodes are applied close to the tooth surface. The compensating electrode 6 is embodied as a cylindrically wound wire at the contact point to the tooth and consequently shields the measuring electrode 5. The reference electrode 4 is placed at any position in the oral cavity. The insulating gel 9 has not yet been applied to the surface of the tooth.

FIG. 5 shows a schematic illustration of the apparatus 1 according to the invention in a longitudinal section: The measuring unit 2 is integrated into the handle 3, and so only one common housing is required. The measuring electrode 5 and the compensating electrode 6 are coupled to one another and placed on the tooth to be examined (the right tooth, illustrated schematically). The measuring electrode 5 and the compensating electrode 6 are embodied as a planar film and can consequently be placed in the interdental space in order to examine the approximal surfaces of the right tooth. The reference electrode 4 is placed at any position in the oral cavity. The insulating gel 9 has not yet been applied to the surface of the tooth.

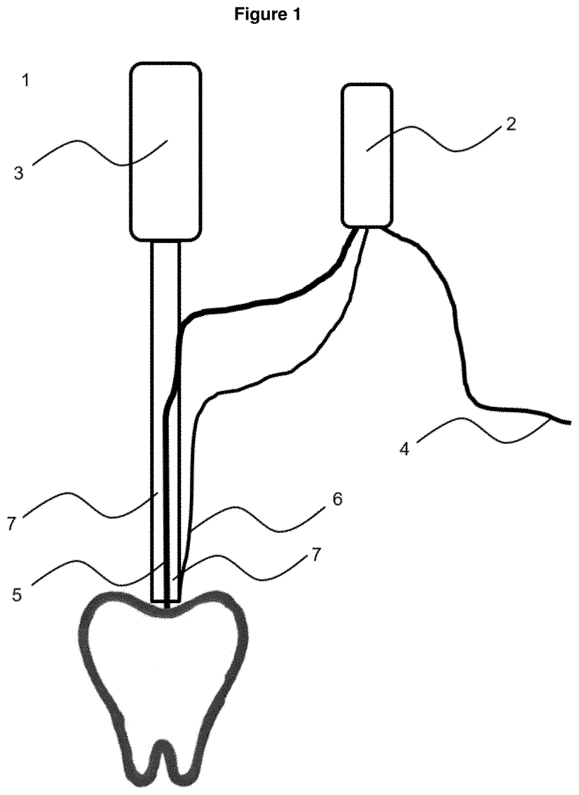

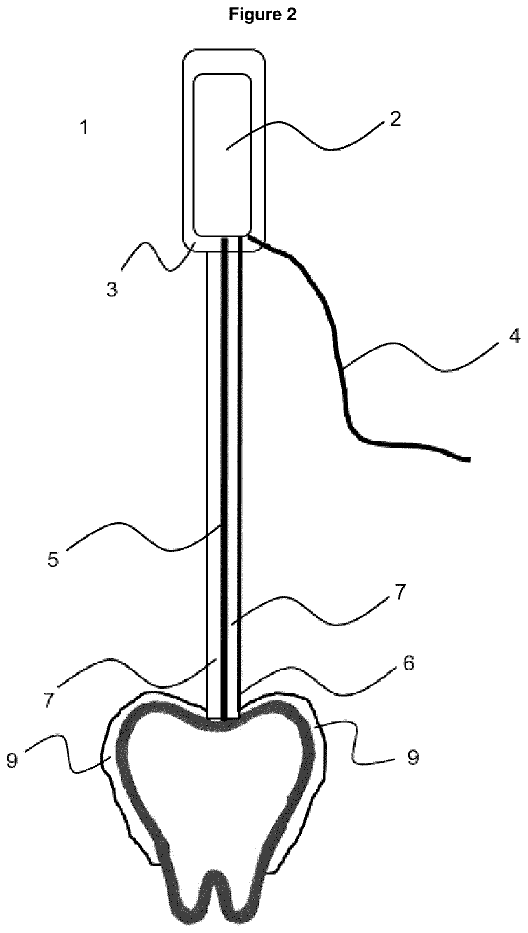

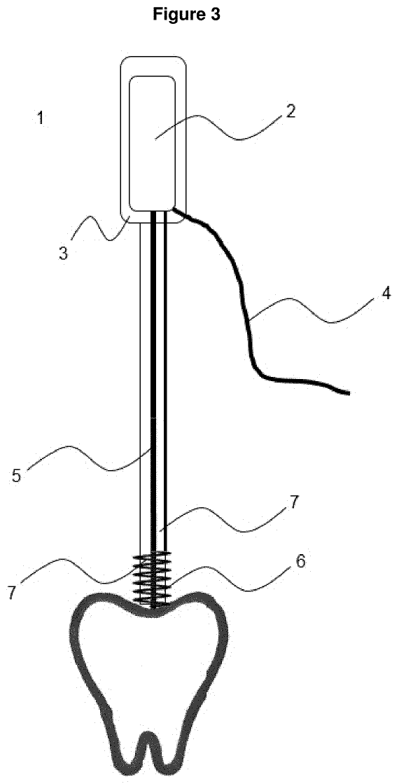

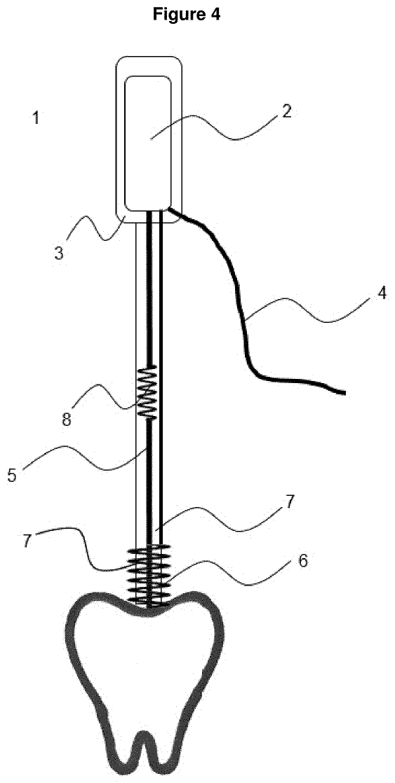

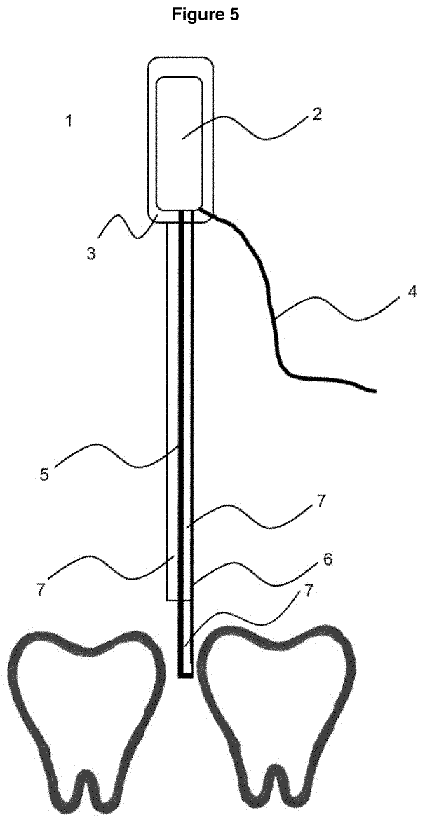

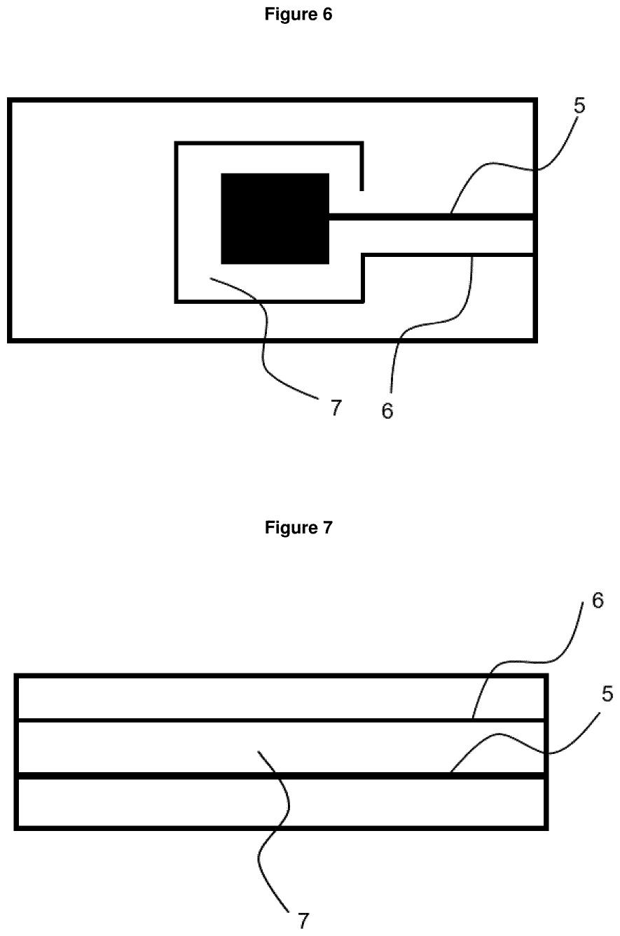

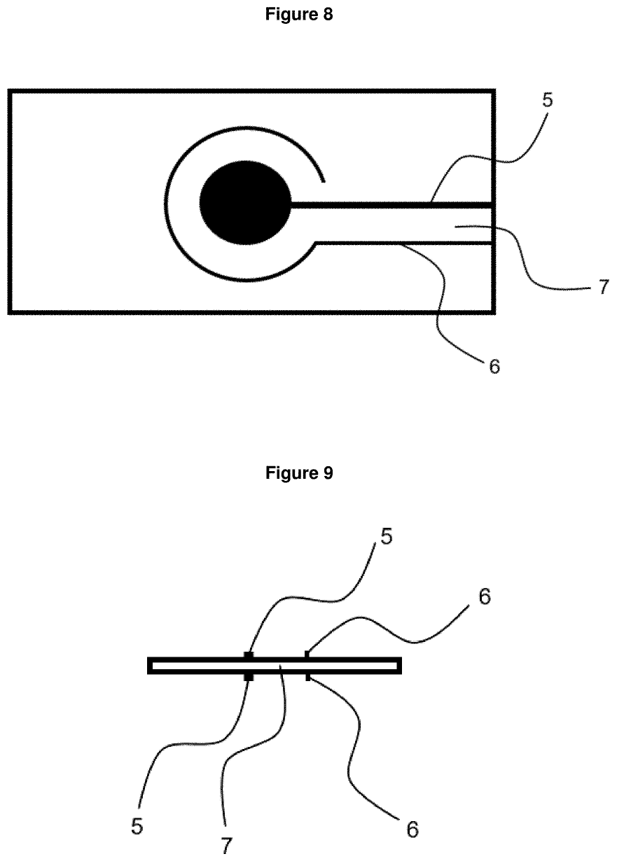

As an exemplary embodiment, FIG. 6 shows a schematic illustration of the measuring electrode 5 and the compensating electrode 6, which are embodied as a planar film. At the same time, the planar film represents insulating layer 7. The measuring electrode 5 is embodied as a planar electrode, which is rectangular in this exemplary embodiment. As a wire-shaped electrode, the compensating electrode 6 is set in the planar film. The compensating electrode 6 is disposed around the rectangular measuring electrode 5 such that it shields the measuring electrode 5.

As an exemplary embodiment, FIG. 7 shows a schematic illustration of the measuring electrode 5 and the compensating electrode 6, which are embodied as a planar film. Both the measuring electrode 5 and the compensating electrode 6 are set in the planar film as straight, wire-shaped electrodes. At the same time, the planar film represents insulating layer 7.

As an exemplary embodiment, FIG. 8 shows a schematic illustration of the measuring electrode 5 and the compensating electrode 6, which are embodied as a planar film. At the same time, the planar film represents insulating layer 7. The measuring electrode 5 is embodied as a planar electrode, which is round in this exemplary embodiment. As a wire-shaped electrode, the compensating electrode 6 is set in the planar film. The compensating electrode 6 is disposed around the round measuring electrode 5 such that it shields the measuring electrode 5.

As an exemplary embodiment, FIG. 9 shows a cross section through a schematic illustration of the measuring electrode 5 and the compensating electrode 6, which are embodied as a planar film. At the same time, the planar film represents insulating layer 7. On the two opposite surfaces of the film, a measuring electrode 5 and a compensating electrode 6 are respectively set in the planar film as straight, wire-shaped electrodes. Suitable programming of the measuring unit 2 causes activation and use for a measurement of either the measuring electrode 5 and the compensating electrode 6 on the one side or the measuring electrode 5 and the compensating electrode 6 on the other side. This allows one planar film to examine two adjacent teeth without having to replace the film.

### LIST OF REFERENCE SIGNS

1 Apparatus according to the invention

2 Measuring unit

3 Handle

4 Reference electrode

5 Measuring electrode

6 Compensating electrode

7 Insulating layer

8 Resilient element for mechanically coupling the compensating electrode 6 to the measuring electrode 5, for example a spring

9 Insulating gel

1. An apparatus for determining the impedance on a tooth, comprising
a measuring unit for producing an AC voltage,
a reference electrode connected to the measuring unit,
a measuring electrode connected to the measuring unit,
a compensating electrode connected to the measuring unit,
wherein the measuring electrode and the reference electrode are disposed relative to one another such that an alternating current can flow on a tooth to be examined between the measuring electrode and the reference electrode in a first position;
and wherein the measuring unit can determine the impedance from the resultant current intensity;
and wherein the measuring electrode and the compensating electrode are at the same electrical potential;
and wherein an insulating layer is applied between the measuring electrode and the compensating electrode to insulate the two electrodes from one another;
and wherein the measuring unit comprises an evaluation unit which captures the change in impedance during a determination at at least two positions of the tooth to be examined;
and wherein the measuring unit is connectable to an output means for outputting the impedance and/or the change in impedance;
and wherein the apparatus comprises a handle for holding the measuring unit during the determination such that the apparatus can be placed on the tooth to be examined during the determination,
characterized in that the apparatus comprises insulating gel, which is disposed in such a way that the measuring electrode is insulated from the saliva in the oral cavity.

2. The apparatus for determining the impedance on a tooth as claimed in claim 1, characterized in that the insulating gel has an electrical resistivity of ρ>500 Ω·m.

3. The apparatus for determining the impedance on a tooth as claimed in claim 2, characterized in that the insulating gel comprises agar and distilled water.

4. The apparatus for determining the impedance on a tooth as claimed in claim 1, characterized in that the insulating gel contains a dye, preferably a blue dye.

5. The apparatus for determining the impedance on a tooth as claimed in claim 1, characterized in that the output means of the measuring unit comprises an optical and/or acoustic and/or haptic indication.

6. The apparatus for determining the impedance on a tooth as claimed in claim 1, characterized in that the reference electrode, the measuring electrode and/or the compensating consist of a metal, such as titanium, silver or iron, or of a metal alloy or, preferably, of stainless steel or carbon.

7. The apparatus for determining the impedance on a tooth as claimed in claim 1, characterized in that the measuring electrode and the compensating electrode are embodied as a planar film.

8. The apparatus for determining the impedance on a tooth as claimed in claim 1, characterized in that the insulating layer consists of a plastic such as polycarbonate or polyethylene, for example.

9. The apparatus for determining the impedance on a tooth as claimed in claim 1, characterized in that the handle has a surface made of electrically insulating material, for example a plastic such as polycarbonate or polyethylene.

10. The apparatus for determining the impedance on a tooth as claimed in claim 1, characterized in that the measuring electrode is mounted in resilient fashion using a resilient element and thereby disposed in axially displaceable fashion in relation to the compensating electrode.

11. The apparatus for determining the impedance on a tooth as claimed in claim 1, characterized in that the measuring electrode has a tapering end.

12. The apparatus for determining the impedance on a tooth as claimed in claim 1, characterized in that the reference electrode is embodied as a bent, stainless wire.

13. The apparatus for determining the impedance on a tooth as claimed in claim 1, characterized in that the measuring unit is integrated in the handle such that only one common housing is required.

14. A method for determining the impedance on a tooth using an apparatus as claimed in claim 1, comprising the steps of:
1. removing the saliva from the tooth to be examined,
2. applying the insulating gel to the tooth to be examined,
3. positioning the measuring electrode in a first position on the tooth to be examined, the measuring electrode being directly incident on the tooth,
4. positioning the reference electrode at any point in the oral cavity,
5. applying an AC voltage between the measuring electrode and the reference electrode using the measuring unit,
6. measuring the resultant current,
7. determining impedance from the AC voltage and the resultant current,
8. repeating steps 3 to 7 at at least one further position on the tooth to be examined,
9. comparing the at least two impedances, whether impedance has changed being captured in the process.

15. The use of an apparatus as claimed in claim 1 for determining the impedance on a tooth.
